(19) United States
(12) Patent Application Publication
(10) Pub. No.: US 2009/0047224 A1
(43) Pub. Date: Feb. 19, 2009

(54) Cosmetic Process for Preventing or Treating the Signs of Skin Ageing or the Orange-Peel Appearance

(75) Inventors: Maria Dalko, Gif s/Yvette (FR); Herve Pageon, Asnieres (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 10/580,230

(22) PCT Filed: Nov. 8, 2004
(86) PCT No.: PCT/EP04/12699

§ 371 Date: Dec. 5, 2006

Related U.S. Application Data
(60) Provisional application No. 60/544,320, filed on Feb. 17, 2004.

(30) Foreign Application Priority Data
Nov. 20, 2003 (FR) 03/50870
Aug. 19, 2004 (FR) 04/08987

(51) Int. Cl. A61K 8/49; A61K 31/4015; A61K 31/4025; C07D 207/444; C07D 407/10

(52) U.S. Cl. 424/59; 514/425; 514/422; 548/542; 548/526

(57) ABSTRACT

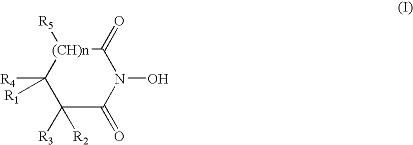

The present invention relates to the use of at least one compound capable of cleaving the diketone crosslinking bonds between two proteins, in a composition applied topically to the skin, as an agent for preventing or treating the signs of ageing of the skin or the orange-peel appearance. The above compound is preferably a N-hydroxy imide of formula (I). These N-hydroxy imides have inhibitory properties on the glycation of proteins and also antioxidant properties. The invention also relates to novel N-hydroxy imides included in formula (I) above.

The present invention relates to the use of at least one compound capable of cleaving the diketone crosslinking bonds between two proteins, such as an N-hydroxy imide, in a composition applied topically to the skin, as an agent for preventing or treating the signs of ageing of the skin or the orange-peel appearance.

It also relates to a cosmetic process for preventing or treating the signs of ageing of the skin or the “orange peel” appearance, comprising the topical application to the skin of a composition comprising, in a physiologically acceptable medium, at least one N-hydroxy imide of given formula.

The invention also relates to novel N-hydroxy imides.

Glycation is a non-enzymatic process involving a saccharide (glucose or ribose) that reacts according to the Maillard reaction with an amino group of an amino acid residue (such as, for example, lysine), particularly an amino acid residue of a protein, to form a Schiff's base. This Schiff's base, after undergoing an Amadori molecular rearrangement, can lead, via a sequence of reactions, to a bridging, particularly an intramolecular bridging such as, for example, of pentosidine type.

This phenomenon is characterized by the appearance of glycation products whose content increases uniformly as a function of age. The glycation products are, for example, pyrraline, carboxymethyl-lysine, pentosidine, crossline, N^{ε}-(2-carboxyethyl)-lysine (CEL), glyoxallysine dimer (GOLD), methyl-glyoxallysine dimer (MOLD), 3DG-ARG imidazolone, versperlysines A, B and C, threosidine or the end products of advanced glycosylation (or AGEs).

The glycation of proteins is thus a universal phenomenon, which is well known in the skin, particularly in its dermal component, and mainly in collagen fibres. Specifically, the glycation of collagen increases uniformly with age, resulting in a uniform increase in the content of glycation products in the skin.

Without wishing to introduce any theory of skin ageing, it should be noted that other changes to collagen which might also be a consequence of glycation, for instance a decrease in heat denaturation, an increase in resistance to enzymatic digestion and an increase in intermolecular bridges, have been able to be demonstrated in the course of skin ageing (Tanaka S. et al., 1988, J. Mol. Biol., 203, 495-505; Takahashi M. et al., 1995, Analytical Biochemistry, 232, 158-162). Furthermore, changes due to the glycation of certain constituents of the basal membrane, for instance collagen IV, laminin and fibronectin, have been able to be demonstrated (Tarsio J F. et al., 1985, Diabetes, 34, 477-484; Tarsio J F. et al., 1988, Diabetes, 37, 532-539; Sternberg M. et al., 1995, C. R. Soc. Biol., 189, 967-985).

Thus, it is understood that, in the course of ageing of the skin, the physicochemical properties of the collagen change and this collagen becomes less readily soluble and less readily degradable. This results in a rigidification of the tissues, essentially leading to a loss of tonicity of the skin.

Moreover, it is very well known that the skin is the result of a close combination between at least two compartments of which it is composed, namely the epidermis and the dermis. The interactions between the dermis and the epidermis are such that it is reasonable to think that a change in one may have consequences on the other. It may thus be suspected that ageing of the dermis, in particular with its glycation phenomena, will inevitably have consequences on the epidermis associated therewith, and that the glycation of collagen must entail changes in the epidermis that necessarily play a part in ageing of the epidermis.

In addition to its effects on ageing of the skin, glycation is involved in the characteristic “orange-peel” appearance of cellulite. Specifically, in cellulite, the glycation of the collagen constituting the majority of the connecting sections results in a rigidification of the tissues, which then imprison the fat globules. The skin thus shows a succession of bumps formed by fatty lumps and of hollows formed by rigidified connecting sections, which are characteristic of the “orange-peel” appearance.

The importance of having available products that reduce or even inhibit the glycation of proteins may thus be appreciated.

Various products capable of inhibiting this glycation reaction are known, including aminoguanidine, which is the inhibitor that is the most widely known (U.S. Pat. No. 5,130,324), taurine (Devamanoharan P. S., 1997, Molecular and Cellular Biochemistry, 177, 245-250), carnosine (Hipkiss A. R., 1995, Febs Letters, 371, 81-85), certain vitamins (B1, B6), bilberry extracts (FR-2 802 425), hydroxystilbenes such as resveratrol (FR-2 796 278) and 3,3′,5,5′-tetrahydroxystilbene (FR-2 802 420) and ergothioneine (FR-2 810 548).

In addition to compounds that inhibit the glycation of proteins, it is known that certain compounds (including the product ALT711 manufactured by Alteon Corporation) are capable of breaking the crosslinking bonds between two proteins, formed as a result of the Maillard reaction (Melton L., Age breakers—Rupturing the body's sugar-protein bond might turn back the clock, Sci. Am., 2000, 283(1): 16; Asif M. et al., An advanced glycation end-product crosslink breaker reverses age-related increases in myocardial stiffness, Proc. Natl. Acad. Sci., 2000, 97(6), 2808-2813).

However, to the Applicant's knowledge, it has never yet been suggested that compounds capable of breaking the crosslinking bonds between two proteins might be useful as anti-glycation agents for topical application to the skin, for the purpose especially of treating the signs of ageing of the skin. It has not been suggested either that N-hydroxy imides were useful for this purpose.

The Applicant has now discovered, surprisingly and unexpectedly, that certain N-hydroxy imides have the property of reducing or even inhibiting the glycation of proteins and thus of acting firstly on the age-related loss of tonicity of the skin, and secondly on the “orange-peel” appearance.

It has moreover been demonstrated that these N-hydroxy imides also have antioxidant properties that allow them to act on the causes of photo-ageing, in addition to their effect on the previously described signs of chronological ageing. They therefore constitute compounds of choice for efficiently combating all the effects of age on the skin, whether of chronological or actinic origin.

One subject of the invention is thus a cosmetic process for preventing or treating the signs of ageing of the skin or the “orange-peel” appearance, comprising the topical application to the skin of a composition comprising, in a physiologically acceptable medium, at least one N-hydroxy imide chosen from (a) the compounds of formula (I):

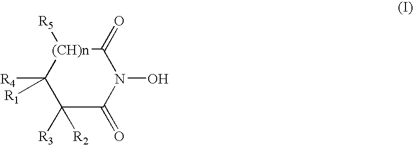

in which:
R₁ and R₂ are each a hydrogen atom or together form a bond;
R₃, R₄ and R₅ independently represent a hydrogen atom; or a linear, branched or cyclic C₁-C₁₂ alkyl radical, or an aryl group or a heterocycle, which may be substituted with one or more groups X chosen from: a C₁-C₆ alkyl radical and a group —OR, —SR, —NRR′, —COOR, —CF₃, —F, —CN, —CH₂OR, and —OCH₂O—, in which R and R′ independently denote a hydrogen atom or a linear or branched C₁-C₄ alkyl radical or an aryl group, it being understood that two adjacent groups X may form a ring with the atoms to which they are attached,
or R₃ and R₄, and/or R₄ and R₅, together form an aliphatic or aromatic ring optionally substituted with one or more alkyl, —ORa, —SRa, —NRaRb or —COORa groups in which Ra and Rb independently denote a hydrogen atom or a linear or branched C₁-C₄ alkyl radical;
n is 0 or 1,
and (b) the organic or mineral salts thereof.

A subject of the invention is also the use of at least one N-hydroxy imide as defined above, in a cosmetic composition comprising a physiologically acceptable medium, as an agent for preventing or treating the signs of ageing of the skin or the “orange-peel” appearance.

Since the N-hydroxy imides according to the invention are capable of cleaving the diketone bridges between two glycated proteins formed as a result of the Maillard reaction, the invention also extends its scope to the use of at least one compound capable of cleaving the diketone crosslinking bonds between two proteins, in a composition applied topically to the skin, as an agent for preventing or treating the signs of ageing of the skin or the orange-peel appearance. The said proteins are generally Amadori products formed as a result of the Maillard reaction.

Examples of alkyl radicals include methyl, ethyl, isopropyl, n-propyl, isobutyl, n-butyl, tert-butyl, pentyl, hexyl, heptyl, octyl, nonyl, decyl, undecyl and dodecyl groups.

A preferred example of an aryl radical is the phenyl radical.

Preferred examples of aliphatic rings that may be mentioned include those containing five or six carbon atoms. A preferred example of an aromatic ring is a phenyl nucleus. Examples of heterocycles that may be mentioned include pyrrole, furan, thiophene, imidazole, imidazolidine, thiazolidine, pyrazole, pyrazolidine, oxazole, oxazolidine, isoxazole, isoxazolidine, isothiazole, isothiazolidine, triazole, triazolidine, oxadiazole, oxadiazolidine, thiadiazole, thiadiazolidine, tetrazole, pyridine, piperidine, pyran and pyrimidine, and hydrogenated derivatives thereof.

Preferred examples of N-hydroxy imides of formula (I) that may be used in the present invention are those for which:
n=0 and R₁═R₂═R₃═R₄═H (N-hydroxysuccinimide) (compound 1 below)
n=0, R₃═R₄═H and R₁ and R₂ together form a bond (N-hydroxymaleimide) (compound 2 below)
n=0, R₃ is a phenyl group, R₄═H and R₁ and R₂ together form a bond (N-hydroxy-2-phenylmaleimide) (compound 3 below)
n=0, R₁ and R₂ together form a bond and R₃ and R₄ together form an aromatic ring (N-hydroxyphthalimide) (compound 4 below)
n=1, R₁ and R₂ together form a bond, R₃ and R₄ together form an aromatic ring and R₄ and R₅ together form an aromatic ring (N,N-(1,8-naphthaloyl)hydroxylamine) (compound 5 below)
n=1 and R₁═R₂═R₃═R₄═R₅═H (N-hydroxy-piperidine-2,6-dione) (compound 6 below).

These compounds are commercially available, especially from the companies Acros and Salor.

Salts of the compound of formula (I) that may be mentioned include the mineral salts, and in particular the sodium, potassium, calcium, magnesium, zinc and strontium salts, and also the organic salts, in particular the triethanolamine salts.

Other examples of compounds corresponding to formula (I) are those identified by the general formula (II):

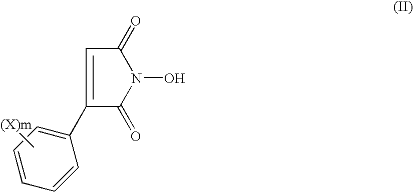

in which:
X independently represents a group chosen from: a C₁-C₆ alkyl radical; or a group —OR, —SR, —NRR′, —COOR, —CF₃, —F or —CN, in which R and R′ independently denote a hydrogen atom or a linear or branched C₁-C₄ alkyl radical or an aryl group; and
m is an integer ranging from 1 to 5,
it being understood that two adjacent groups X on the ring may together form an —OCH₂O— bond.

Preferably, m is equal to 1 or 2. When m is equal to 1, the substituent X may be in an ortho, meta or para position relative to the N-hydroxymaleimide. When m is equal to 2, the substituents X are preferably each in a meta position relative to the N-hydroxy-maleimide.

To the Applicant's knowledge, these compounds are novel, such that the present invention also relates to these compounds per se.

Examples of compounds of formula (II) are those corresponding to the chemical formulae (a) to (k) below:

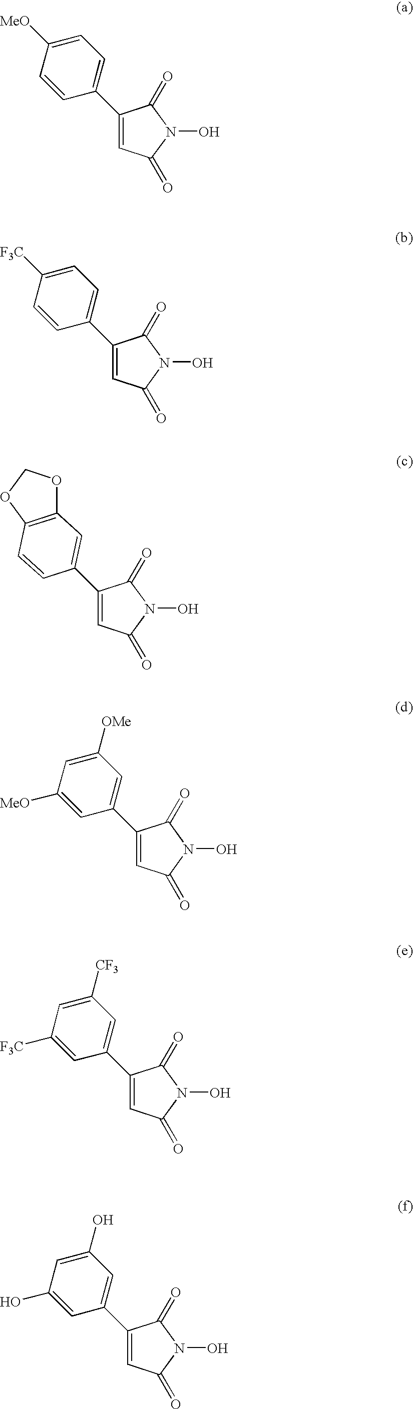

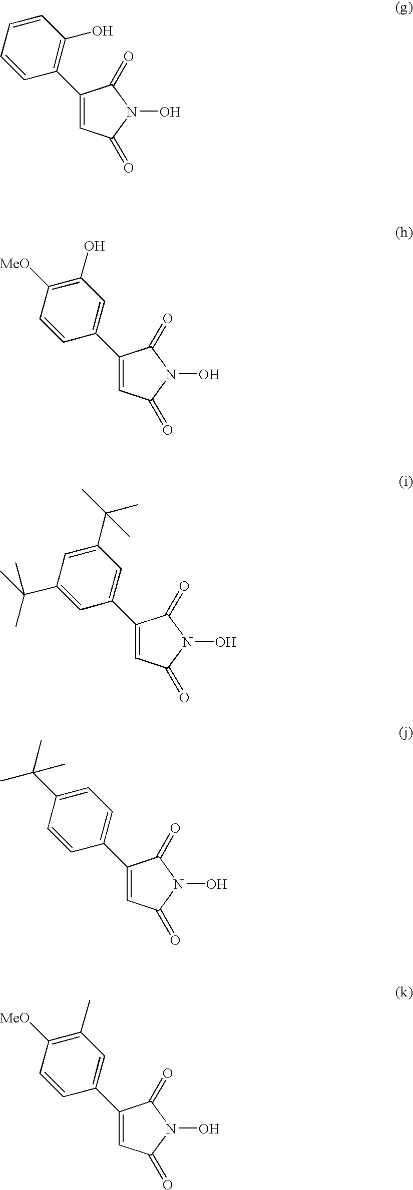

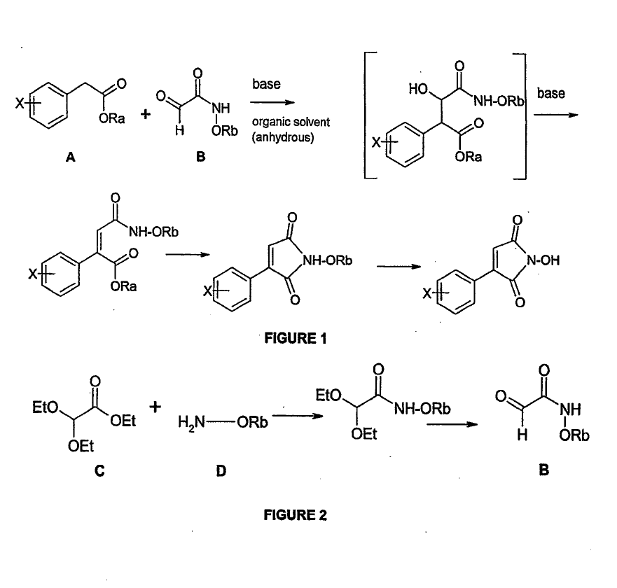

The compounds of formula (II) may be prepared by synthesis, for example according to the process illustrated in the attached FIG. 1.

As indicated in FIG. 1, a commercially available phenethylic acid derivative A, in which Ra denotes a hydrogen atom or a methyl, ethyl, isopropyl, tert-butyl or benzyl group, is reacted with a compound B in which Rb denotes a protecting group such as a benzyl group, in the presence of a base in an anhydrous organic medium, for example in the presence of sodium hydride in a THF/DMF mixture or of potassium carbonate in acetone. The compound of formula (II) is obtained spontaneously or by saponification of the intermediate obtained using a base such as potassium carbonate or sodium hydroxide in a water/acetone mixture.

Compound B may be prepared, as indicated in FIG. 2, from commercial compounds C and D. After saponification of the ester function of compound C (for example using a base such as potassium carbonate or sodium hydroxide in a water/acetone mixture), the isolated acid is activated according to a standard activation method (DCC or CDI) and is then reacted with the hydroxylamine D, for example in anhydrous dichloromethane. An acetal is obtained, which is then deprotected by acidic treatment (for example with a catalytic amount of p-toluenesulfonic acid or alternatively via a water/acetic acid treatment) to obtain compound B.

A subject of the invention is also a composition containing, in a physiologically acceptable medium, at least one compound of formula (II) above. This composition is preferably suitable for topical application to the skin.

As demonstrated in the examples below, the compounds of formula (I) according to the invention—including the compounds of formula (II)—have the property of inhibiting the glycation of dermal proteins, and also antioxidant properties, such that the process according to the invention is in particular intended to prevent or treat the signs of ageing of the skin associated with the glycation of dermal proteins, especially to prevent or treat the loss of tonicity or elasticity of the skin and/or to prevent or combat the signs of photo-ageing, in particular wrinkles and/or pigmentation marks. As a variant, it may be performed to combat other skin conditions resulting from glycation of proteins, such as the “orange-peel” appearance accompanying cellulite.

The composition used according to the invention is thus preferably applied to individuals presenting a lack of tonicity or elasticity of the skin and/or signs of photo-ageing, in particular wrinkles and/or pigmentation marks, and/or cellulite.

This composition contains an amount of N-hydroxy imide that is sufficient to obtain the desired effect; for example, the said N-hydroxy imide represents from 0.005% to 15%, preferably from 0.01% to 5% and more preferably from 0.1% to 2% of the total weight of the composition.

The composition may be in any galenical form conventionally used for topical application, and especially in the form of an aqueous gel or an aqueous or aqueous-alcoholic solution. By adding a fatty or oily phase, it may also be in the form of a dispersion of the lotion or serum type, an emulsion of liquid or semi-liquid consistency of the milk type, obtained by dispersing a fatty phase in an aqueous phase (O/W) or conversely (W/O), or a suspension or emulsion of soft, semi-solid or solid consistency of cream or gel type, or alternatively a multiple emulsion (W/O/W or O/W/O), a microemulsion, a vesicular dispersion of ionic and/or nonionic type, or a wax/aqueous phase dispersion. These compositions are prepared according to the usual methods.

According to one preferred embodiment of the invention, the composition is in the form of an emulsion.

The composition used according to the invention may be more or less fluid and may have the appearance of a white or coloured cream, an ointment, a gel, a milk, a lotion or a serum. For use in the treatment of the signs of ageing of the skin, it is preferred to use a composition in the form of a cream or a serum. For use in the treatment of the “orange-peel” appearance, the composition according to the invention may optionally be applied in the form of a stick or in the form of an aerosol, or alternatively in the form of a liquid to be sprayed forming a patch on the skin.

When the composition is in the form of an emulsion, the proportion of the oily phase of the emulsion may range, for example, from 5% to 80% by weight and preferably from 5% to 50% by weight relative to the total weight of the composition. The oils, emulsifiers and co-emulsifiers used in the composition in emulsion form are chosen from those conventionally used in cosmetics or dermatology. The emulsifier and the co-emulsifier are generally present in the composition in a proportion ranging from 0.3% to 30% by weight and preferably from 0.5% to 20% by weight relative to the total weight of the composition. The emulsion may also contain lipid vesicles.

As fatty substances that may be used in the invention, it is possible to use oils and especially mineral oils (liquid petroleum jelly), oils of plant origin (avocado oil or soybean oil), oils of animal origin (lanolin), synthetic oils (perhydrosqualene), silicone oils (cyclomethicone) and fluoro oils (perfluoropolyethers). Fatty alcohols such as cetyl alcohol, fatty acids, waxes and gums, and in particular silicone gums, may also be used as fatty substances.

As emulsifiers and co-emulsifiers that may be used in the invention, examples that may be mentioned include fatty acid esters of polyethylene glycol, such as PEG-100 stearate, PEG-50 stearate and PEG-40 stearate; fatty acid esters of polyols, such as glyceryl stearate, sorbitan tristearate and the oxyethylenated sorbitan stearates available under the trade names Tween® 20 or Tween® 60, for example; and mixtures thereof.

The composition according to the invention may also contain adjuvants that are common in cosmetics and dermatology, such as hydrophilic or lipophilic gelling agents, active agents, preserving agents, solvents, fragrances, fillers, pigments, odour absorbers and dyestuffs. The amounts of these various adjuvants are those conventionally used in the fields under consideration, for example from 0.01% to 20% of the total weight of the composition. Depending on their nature, these adjuvants may be introduced into the fatty phase or into the aqueous phase.

These adjuvants, and the concentrations thereof, should be such that they do not harm the advantageous properties of the N-hydroxy imides according to the invention.

Hydrophilic gelling agents that may be mentioned in particular include carboxyvinyl polymers (carbomer), acrylic copolymers such as acrylate/alkylacrylate copolymers, polyacrylamides, polysaccharides, natural gums and clays, and lipophilic gelling agents that may be mentioned include modified clays, for instance Bentones, metal salts of fatty acids and hydrophobic silica.

As active agents, the composition according to the invention may comprise at least one compound chosen from: desquamating agents and/or moisturizers (such as α- and β-hydroxy acids, in particular 5-n-octanoylsalicylic acid, ceramides, hyaluronic acid, 2-oxothiazolidine-4-carboxylic acid, HEPES, honey and glycerol); depigmenting agents (including vitamin C and derivatives thereof such as ascorbyl glucoside); agents for stimulating fibroblast proliferation (including soybean extracts); agents for stimulating keratinocyte proliferation (including retinoids such as retinol); agents for stimulating keratinocyte differentiation (including lupin and maize extracts); dermo-decontracting agents (such as adenosine, alverine and wild yam extracts); tensioning agents (including colloidal silica, mixed silicates and acrylic-silicone latices); anti-pollution agents or free-radical scavengers (including vitamin E and coenzyme Q10); and lipolytic active agents or active agents for reducing adipose tissue (including caffeine and Ginkgo biloba extracts).

Advantageously, the composition according to the invention also contains at least one UVA-active and/or UVB-active organic photo-protective agent and/or a mineral photoprotective agent.

As organic photoprotective agents that may be used in the composition according to the invention, mention may be made of the following screening agents, mentioned under their CTFA name or chemical name, depending on the case: ethylhexyl salicylate, ethyl-hexyl methoxycinnamate, Octocrylene, phenylbenzimid-azolesulfonic acid, Benzophenone-3, Benzophenone-4, Benzophenone-5,4-methylbenzylidenecamphor, terephthalylidenedicamphorsulfonic acid, disodium phenyldibenzimidazoletetrasulfonate, 2,4,6-tris-(diisobutyl 4′-aminobenzalmalonate)-β-triazine, Anisotriazine, ethylhexyl triazone, diethylhexyl butamidotriazone, methylenebis-benzotriazolyl-tetra-methylbutylphenol, drometrizole trisiloxane, 1,1-dicarboxy(2,2′-dimethylpropyl)-4,4-diphenylbutadiene, and mixtures thereof.

Examples of mineral photoprotective agents that may be mentioned include pigments or nanopigments (mean size of the primary particles: generally between 5 nm and 100 nm and preferably between 10 nm and 50 nm) of metal oxides, for example nanopigments of titanium oxide (amorphous or crystallized in rutile and/or anatase form), iron oxide, zinc oxide, zirconium oxide or cerium oxide. These pigments may be uncoated or coated with alumina and/or aluminium stearate. Such coated or uncoated metal oxide nanopigments are described in particular in patent applications EP 518 772 and EP 518 773.

The invention will now be illustrated by means of the non-limiting examples that follow.

### EXAMPLES

### Example 1

### Demonstration of the Anti-Glycation Effect

A solution of bovine serum albumin at 5 or 10 mg/ml dissolved in phosphate-buffered saline is incubated at 37° C. for four weeks in the presence or absence of the test compounds at a concentration of between 20 and 160 μg/ml, and in the presence or absence of D-ribose at 100 mM.

After this incubation, dialysis of each sample is performed for 24 hours against MilliQ water.

The glycation of the BSA is evaluated by measuring the fluorescence of each sample at λex 320/λex 380 nm (which corresponds to the formation of the pentosidine glycation product) and at λex 370/λex 440 nm (which corresponds to AGEs).

The inhibition of glycation corresponds to a decrease in fluorescence compared with the glycated control (treated with sugar). The concentration of compound leading to a 50% inhibition of glycation, or IC₅₀, is determined.

Aminoguanidine is used as positive control and tested at various concentrations of between 20 and 160 μg/ml.

The results are given in Table 1 below.

TABLE 1

| | | |
|---|---|---|
| | λex 320/λex 380 nm | λex 370/λex 440 nm |
| Product | IC₅₀ | IC₅₀ |

| | | | | |
|---|---|---|---|---|
| Compound 2 | 800 | μM | 600 | μM |
| Compound 3 | 30 | μM | 40 | μM |
| Compound 4 | 200 | μM | 250 | μM |
| Compound 5 | 0.8 | μM | 45 | μM |
| Aminoguanidine | 200 | μM | 700 | μM |

This test thus demonstrates the glycation-inhibiting effect of the N-hydroxy imides according to the invention.

Compounds 1 and 6 also have anti-glycation activity, but more moderate than aminoguanidine. However, compound 1 has the advantage of not releasing ammonia on heating, in contrast with aminoguanidine, which makes it more suitable for manufacturing cosmetic compositions.

### Example 2

### Demonstration of the Antioxidant Effect

The DNA-protecting effect of the compounds according to the invention was evaluated by comparison with vitamin C and N-acetylcysteine, which are two well-known antioxidants.

In this test, supercoiled circular DNA (plasmid pBR322, Roche) is placed in solution in the presence of 10 mM phosphate buffer (pH=7) and ferric ions (FeCl₃, Sigma) at a concentration of 1 μM and in the presence of riboflavin (Sigma) at a concentration of 0.5 μM. The iron amplifies, via the Fenton reaction, the photo-oxidative impact on the DNA. Its presence also makes it possible to evaluate the potential chelating effect of the N-hydroxy imides according to the invention.

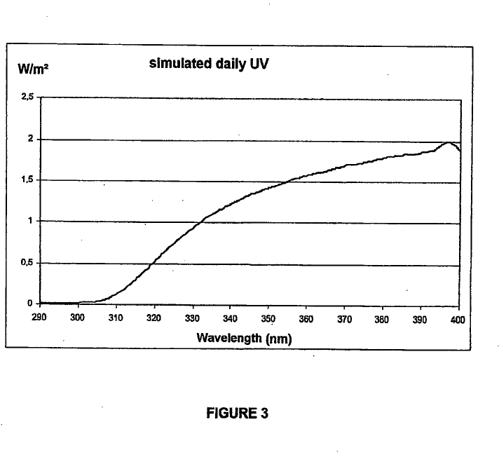

The samples are exposed for 30 minutes to simulated daily UV (attenuated UVB+total UVA) obtained using the Oriel solar simulator equipped with a suitable optical filter and a dichroic mirror. The corresponding spectrum is illustrated in FIG. 3.

The DNA is then subjected to electrophoresis on 1% agarose gel to separate the supercoiled forms, relaxed due to induction of breaks and linearized. After treatment in a gel with the fluorescent intercalating agent ethidium bromide, these three forms may be quantified by densitometry of the agarose gel under UV. The level of protection is evaluated as follows:

The score (0) means that the protection provided is not significant.
The score (+) means that the active agent reduces the photodegradation of the DNA by at least 20%.
The score (++) means that the active agent reduces the photodegradation of the DNA by at least 50%.
The score (−) means that the test product was found to be photoreactive and increased the level of breaks in the DNA under UV.

The results obtained are collated in Table 2 below:

TABLE 2

| | | |
|---|---|---|
| Compound | Concentration | Score |

| | | | |
|---|---|---|---|
| 3 | 0.1 | mM | ++ |
| 5 | 0.01 | mM | + |
| Vitamin C | 1 | mM | + |
| N-Acetylcysteine | 1 | mM | ++ |

As is seen from this table, the compounds according to the invention were found to be as active as, or even more active than, the standard antioxidants.

### Example 3

### Cosmetic Composition

### O/W Cream

The composition below is prepared in a conventional manner for those skilled in the art. The percentages indicated are weight percentages.

| | |
|---|---|
| Compound 3 | 1% |
| Glyceryl stearate | 2% |
| Oxyethylenated sorbitan monostearate | 1% |
| (20 EO) | |
| Stearic acid | 1.4% |
| Triethanolamine | 0.7% |
| Carbomer | 0.4% |
| Liquid fraction of shea butter | 12% |
| Perhydrosqualene | 12% |
| Antioxidant | 0.05% |
| Fragrance | 5% |
| Preserving agents | 0.3% |
| Water | qs 100% |

This cream may be applied to the face morning and/or evening to prevent or combat the signs of ageing of the skin (wrinkles or loss of tonicity of the skin).

1. A process of preventing or treating signs of ageing of skin or an orange-peel appearance comprising:
applying topically to the skin a composition, in a physiologically acceptable medium, comprising at least one compound capable of cleaving the diketone crosslinking bonds between two proteins.

2. The process according to claim 1, wherein the proteins are Amadori products formed as a result of the Maillard reaction.

3. The process according to claim 1, wherein the compound capable of cleaving the diketone crosslinking bonds between two proteins is a N-hydroxy imide chosen from (a) the compounds of formula (I): 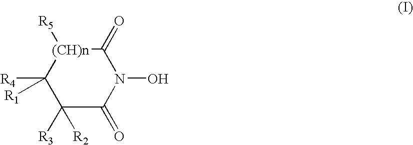
wherein:
R₁ and R₂ are each a hydrogen atom or together form a bond;
R₃, R₄ and R₅ independently represent a hydrogen atom; or a linear, branched or cyclic C₁-C₁₂ alkyl radical, or an aryl group or a heterocycle, which may be substituted with one or more groups X chosen from: a C₁-C₆ alkyl radical and a group —OR, —SR, —NRR′, —COOR, —CF₃, —F, —CN, —CH₂OR, and —OCH₂O—, in which R and R′ independently denote a hydrogen atom or a linear or branched C₁-C₄ alkyl radical or an aryl group, it being understood that two adjacent groups X may form a ring with the atoms to which they are attached,
or R₃ and R₄, and/or R₄ and R₅, together form an aliphatic or aromatic ring optionally substituted with one or more alkyl, —ORa, —SRa, —NRaRb or —COORa groups in which Ra and Rb independently denote a hydrogen atom or a linear or branched C₁-C₄ alkyl radical;
n is 0 or 1,
and (b) the organic or mineral salts thereof.

4. The process according to claim 3, wherein n=0 and R₁═R₂═R₃═R₄═H.

5. The process according to claim 3, wherein n=0, R₃═R₄═H and R₁ and R₂ together form a bond.

6. The process according to claim 3, wherein n=0, R₃ is a phenyl group, R₄═H and R₁ and R₂ together form a bond.

7. The process according to claim 3, wherein n=0, R₁ and R₂ together form a bond and R₃ and R₄ together form an aromatic ring.

8. The process according to claim 3, wherein n=1, R₁ and R₂ together form a bond, R₃ and R₄ together form an aromatic ring and R₄ and R₅ together form an aromatic ring.

9. The process according to claim 3, wherein n=1 and R₁═R₂═R₃═R₄═R₅═H.

10. (Canceled)

11. The process according to claim 1, wherein the N-hydroxy imide represents from 0.1% to 2% of the total weight of the composition.

12. The process according to claim 1, for preventing or treating the signs of ageing of the skin associated with the glycation of dermal proteins.

13. The process according to claim 1, for preventing or treating the loss of tonicity or elasticity of the skin.

14. The process according to claim 1, for preventing or combating the signs of photo-ageing of the skin.

15. The process according to claim 14, for preventing or combating wrinkles and/or pigmentation marks.

16. The process according to claim 1, wherein the composition is applied to individuals with a lack of tonicity or elasticity of the skin.

17. The process according to, claim 1, wherein the composition is applied to individuals with cellulite.

18. The process according to claim 1, wherein the composition is applied to individuals presenting signs of photo-ageing.

19. Compounds of general formula (II): 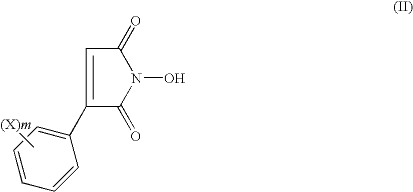
wherein:
X independently represents: a C₁-C₆ alkyl radical; or a group —OR, —SR, —NRR′, —COOR, —CF₃, —F or —CN, in which R and R′ independently denote a hydrogen atom or a linear or branched C₁-C₄ alkyl radical or an aryl group; and
m is an integer ranging from 1 to 5,
it being understood that two adjacent groups X on the ring may together form an —OCH₂O—bond.

20. The compound according to claim 19, corresponding to one of the following chemical formulae (a) to (k):

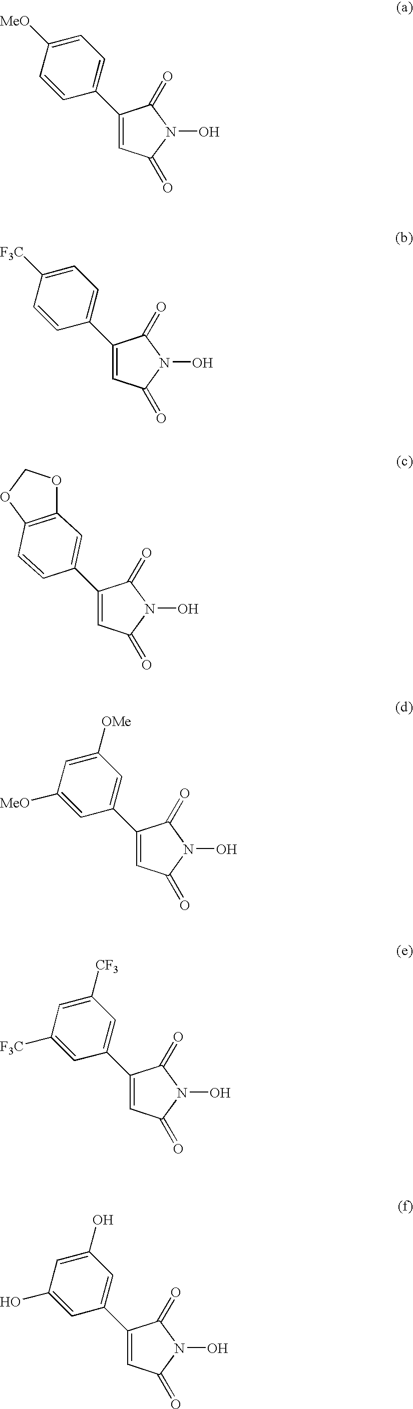

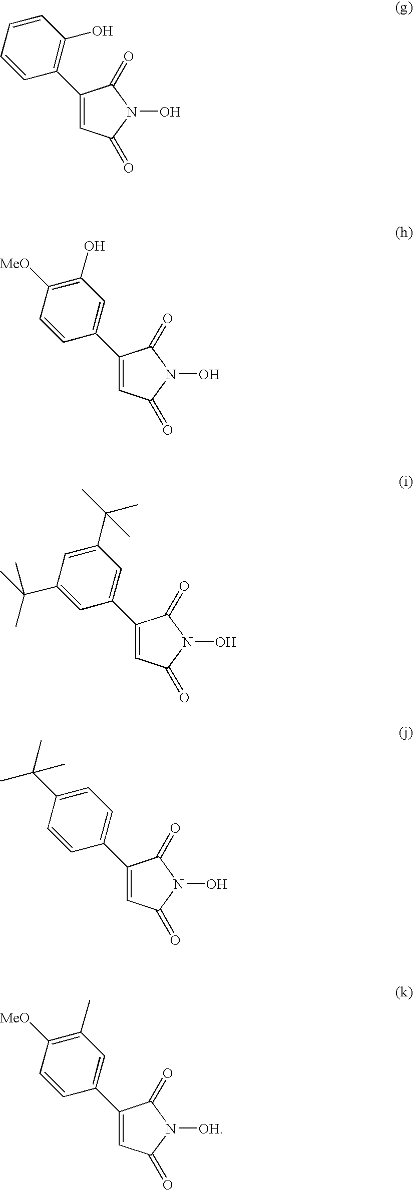

21. A composition comprising, in a physiologically acceptable medium, at least one compound according to claim 19.

22. The composition according to claim 21, for topical application to the skin.

23. The composition according to claim 21, further comprising at least one compound selected from the group consisting of: desquamating agents and/or moisturizers; depigmenting agents; agents for stimulating fibroblast proliferation; agents for stimulating keratinocyte proliferation; agents for stimulating keratinocyte differentiation; dermo-decontracting agents; tensioning agents; anti-pollution agents or free-radical scavengers; and lipolytic active agents or active agents for reducing adipose tissue.

24. The composition according to claim 21, further comprising at least one UVA-active and/or UVB-active organic photo-protective agent and/or a mineral photo-protective agent.
